(19) United States
(12) Patent Application Publication
(10) Pub. No.: US 2022/0354440 A1
(43) Pub. Date: Nov. 10, 2022

(54) Worldwide vision screening and visual field screening booth, kiosk, or exam room using artificial intelligence, screen sharing technology, and telemedicine video conferencing system to interconnect patient with eye doctor anywhere around the world via the internet using ethernet, 4G, 5G, 6G or Wifi for teleconsultation and to review results

(71) Applicant: Willis Dennis Grajales, LITTLE ELM, TX (US)

(72) Inventor: Willis Dennis Grajales, LITTLE ELM, TX (US)

(21) Appl. No.: 17/307,901

(22) Filed: May 4, 2021

(51) Int. Cl. A61B 5/00; G06N 20/00; G16H 50/30; G16H 80/00; A61B 3/113; A61B 3/032; A61B 3/024; A61B 3/06; A61G 10/00; A61L 2/10

(52) U.S. Cl. CPC A61B 5/7465; G06N 20/00; G16H 50/30; G16H 80/00; A61B 3/113; A61B 3/032; A61B 3/024; A61B 3/066; A61G 10/00; A61L 2/10; A61L 2202/11; A61L 2202/14; A61L 2202/17; A61L 2202/25

(57) ABSTRACT

The present disclosure describes the clinical workflow, method, apparatus and system of an on demand artificial intelligence visual screening and visual field screening system that incorporates a display simulator cockpit frame system inside a booth, kiosk, or exam room with machine learning and telemedicine capabilities. According to various embodiments, an artificial intelligent, physical or virtual assistant may help in the screening process of the patient, and an eye doctor via telemedicine may connect to the computers in display simulator cockpit system to perform necessary medical consultations, visual examination or screenings via the display simulator cockpit frame set-up display system. Where an on demand health care provider via remote administration tool technology, remote screen sharing and remote control software control the medical equipment, provide medical consultations and medical examination to a patient from anywhere in the world via cellphone wireless networks or wifi to interconnect both systems.

### RELATED APPLICATIONS

This U.S. patent application is a continuation of U.S. Provisional Application No. 63/165,085 filed Mar. 23, 2021 titled “A worldwide vision screening and visual field screening booth, kiosk, or exam room using artificial intelligence, screen sharing technology, and telemedicine video conferencing system to interconnect patient with eye doctor anywhere around the world via the internet using ethernet, 4G, 5G, 6G or Wifi for teleconsultation and to review results.”.

### DETAILED DESCRIPTION OF EMBODIMENTS OF THE INVENTION

The present invention provides a method, system methods, apparatus, and workflows of a vision screening technology with artificial intelligence machine learning (AIML) and telemedicine vision screening technology. The following is a booth, kiosk, or exam room with a simulator cockpit frame installed in a plurality of locations worldwide. (FIG. 2) (FIG. 14; 511). The booth, kiosk, or exam room have a simulator cockpit frame with mounted computers systems running artificial intelligence software, machine learning software, visual acuity screening software, kiosk software, visual field screening software, simulator cockpit frame adapters, exam chair, furniture, large screen monitor, small screen monitors, eye tracking device, internet, e-commerce webpage, electronic medical screening record, UV disinfecting system, walls, roof, air vents, (smart glass doors or smart glass windows), (RAT) remote administration tool, remote desktop software and telemedicine software technology in a mobile or non-mobile location (AI-VFTR) (Artificial intelligent visual field telemedicine room). (FIG. 3; 7, 8) (FIG. 14; 34, 16, 43, 33, 35, 503, 24, 511). The outside of the room has sign-in computers runs that may run kiosk software with e-commerce payments and gather patient information (user medical history information) inputted by any (user). (FIG. 3; 11) (FIG. 2; 7).

The (user) may be a patient, customer, or any person needing a medical screening. The payments portal can direct the (user) to pay via credit card shopping cart, self-checkout, central bank digital currencies, or cryptocurrency address if needed for the screening service to be rendered. (FIG. 3; 12). In some cases, the payment can be waived as a no-cost service in humanitarian missions or free screenings. The rooms may be equipped with smart glasses, doors, and windows. (FIG. 12; 550, 10). The doors with (smart glass) or (smart film) are used for privacy and reducing brightness in the room. The smart glass doors can be activated remotely or non remotely to adjusting light transmission between transparent, opaque, or electrochromic using an AC power smart switch.

The (SG) smart glass can be activated automatically remotely to prevent brightness during the screenings. The smart glass will remain activated after completion of the vision screening, and the room will turn on UV disinfecting lights to disinfect the environment before the next (user) may enter the exam room. (FIG. 13; 201). The UV disinfecting system may be attached to the room or maybe attached to the cockpit simulation frame adapter. Most of the equipment (clicker and virtual reality headset) will have an enclosed cabinet attached to the simulator cockpit frame with a UV light that can become activated after every encounter to disinfect the equipment. (FIG. 14; 43, 201). After the new (user) may enter the room, he or she will be provided a set of alcohol prep pads and cleaning wipes to clean the chair and clean the tablet, virtual reality headset, clicker, and equipment. (FIG. 14).

The (AI-VFTR) can be used by a (user) requiring an on-demand vision screening and visual field screening. The screening is done via automated vision screening software running on the computer systems. All the computers or tablets that run inside (on the simulator cockpit frame system) and outside of the (AI-VFTR) may be running on any operating system (windows, android, IOS, Linux, raspberry pi, chrome os) depending on software and system used. (FIG. 5; 30, 33, 34). In the following computers used, the image will be (screen mirrored). Screen mirroring may be achieved via HDMI, wifi, or Bluetooth to the large and smalls screen smart tv or display monitor attached to the simulator cockpit frame. (FIG. 10; 509).

The computers that operate inside the (booth, kiosk, or exam room) but are attached to the simulator cockpit frame. (FIG. 14; 511). In cases of deficiencies in the vision screening, the (user) can also connect to network telemedicine on-demand healthcare providers (HCP). A network health care provider (HCP) is an individual health care professional or a health facility organization licensed to provide health care diagnosis, treatment services, including medication, medical device prescriptions, and medical devices. The connection to the health care provider will be via a live telemedicine consultation using video audio and audio call via cellphones. The real-time audio call will be via the (user's) personal cellphone.

If a live consultation is performed, all of the equipment inside the exam room can be controlled or viewed by a health care provider from any remote location via screen and control software (RAT), remote administration tool technology (RDT), remote access technology, and cloud computing technology. This allows the healthcare provider to store, access (user) information, and update records remotely if needed. The on-demand health care practitioner can be any health care practitioner specialized or trained in the area needed for the neurologic, optometric, ophthalmologic vision screening evaluation. Most of the time, the on-demand health care practitioner (HCP) will be an Optometrist or Ophthalmologist.

The use of the simulator cockpit system inside the booth, kiosk, or exam room is to provide vision health, vision screening, and visual field screening to underserved people or emergency room patients needing such services anywhere in the world (FIG. 2). Visual field loss may occur due to many diseases or disorders of the eye, optic nerve, or brain. (FIG. 9A; 73, 1, 2, 3, 4, 29). All the telemedicine services can be provided by a network of health care providers with valid licenses that may connect to the computer system from anywhere in the world. The use of remote (SS) screen sharing technology and telemedicine video conferencing system to interconnect patient with a health care provider (Eye doctor) anywhere around the world via satellite internet, ethernet, 4G, 5G, 6G or Wifi for tele-consultation and to review vision screening results with patients. (FIG. 6; 21, 76, 80, 14).

An assistant will help with the instruction and guide patients or customers to sign in, pay and start the service. (FIG. 4; 202, 200). The (user) will receive a text message or email with a QR code that will be used to unlock the exam room door. (FIG. 4; 70, 601, 14, 202, 200, 10). The (Assistant) can be an assistant technician, virtual assistant technician (AT), or (AIVA) an Artificial intelligent virtual assistant. In some cases, a (remote health care provider) may be an assistant). An (AIVA) artificial intelligent assistant is a computer software virtual assistant (AIVA).

A virtual assistant is a human assistant with remote access control and video audio capabilities from a remote location to provide service at the site. (FIG. 2; 200, 202, 6) (FIG. 5; 200). The virtual assistant may also use a cellphone network and line (phone call) to access a connection with the (user) in the room. A physical assistant technician is an optometric assistant, ophthalmic assistant, optician assistants, medical assistants who help eye doctors in vision clinics. The (AIVA) Artificial intelligent visual assistant is a software agent that can perform tasks or services for an individual based on commands or questions.

The (AIVA) will be able to interpret human speech and respond via synthesized voices. The assistant will provide instruction as soon as a person requests a service via a doorbell or link on the sign-in computer. The customer or patient can request the service via an online portal or the computers outside of the room. The (assistant) will instruct the patient or customer to fill out the (user) medical history information on a computer outside of the visual screening booth, kiosk, or exam room, or on the patient webpage linked to a QR code on the wall. The (user) may scan the QR code that directs them to a mobile application or webpage to fill out their medical information. (FIG. 2; 70, 7, 11) (FIG. 3; 12, 11).

All information is stored in a secure encrypted database that can be accessed by the (assistant) remotely or by a health care provider. (FIG. 3). The assistant will ensure the (user) selects the desired vision screenings options and visual field screenings options on the computers outside of the visual field screening booth, kiosk, or exam room. After the (user) pays for the service, the exam room door will automatically open or be opened or unlocked remotely. The assistant will instruct the (user) to use one of the eye patches and sit on the exam chair. (FIG. 5; 14, 15, 35) (FIG. 6) (FIG. 7). The patient will be instructed to cover his right eye with a patch first and the change to the left eye after the test is completed.

A large screen directly in front of the exam chair will display vision screening tests. The vision screening and visual field screening will start when the (user) is ready and presses the ready button on the tablet. The (user) will finish each session of the vision screening exam, and the computer software will use artificial intelligence software (AIS) and machine learning (AIML) (ML) software to determine if any deficiencies were noticed. (FIG. 7; 400, 44, 401). The computers in the exam room will also provide possible determinate results noted via image search and machine learning. (FIG. 8).

All results will be inputted into a database by the assistant, health care provider, or (AIVA) AI assistant. After the results are gathered, the assistant (virtual, AI, or physical) will ask the (user) if he or she would like to talk to a live eye doctor (health care provider) via a telemedicine virtual connection. The (assistant) can also ask the patient if he or she would like to be referred to a list of health care providers in the area and provide a list. If the patient accepts the extra charge for the live telemedicine consultation, an health care provider will appear on the screen to go over the patient's results and recommend a referral or consultation if needed. (FIG. 13; 205, 77) (FIG. 13A; 42, 76, 77).

The health care provider will asks the (user) if they would like to have an additional, more precise virtual reality visual field test. (FIG. 10; 77, 80, 601, 600, 43). This visual field test is used for further review for an additional cost or no cost. The assistant will communicate with a health care provider (Neurologist, primary care physician, optometrist, or ophthalmologist) within the network to review the visual field results when completed. The assistant and or (health care provider) (eye doctor) will email or print the vision screening and visual field screening report for the (user). (FIG. 11; 503, 600, 400, 503, 44). The screening report may also be emailed to the (user).

The (AI-VFTR) (Artificial intelligent visual field telemedicine room) system exam room can be of any size or dimension and can accommodate one, two, or three standing or sitting persons (FIG. 5). The exam room may be a room inside a building or a booth similar to a photo booth. The exam room may or may not have smart glass technology, depending on the design used. The exam rooms may be freestanding or non-freestanding with dividers or without dividers depending on the design used. The booth, kiosk, or exam room may have multiple designs and models. (FIG. 12; 10, 550, 551, 81, 35, 700, 82, 46, 47, 82, 48) (FIG. 5) (FIG. 4).

The exam room can be rooms inside or outside of any building or structure. The exam rooms can also be placed side by side, providing multiple exam rooms in one location. The exam rooms with the simulator cockpit frame system can be installed in any room. The system can be used in any common room by adding a simulator cockpit frame system. The rooms can be inside hospitals, malls, stores, grocery stores, DMV, schools, medical offices, or any mobile or non-mobile location.

An (AI assistant) assistant or virtual assistant may be inside or outside of the room (FIG. 5; 200). The following will have a laptop or tablet outside of the room in which the (user) can input his or her (user) information and medical history (FIG. 2; 7, 6, 9). The outside of the room may have a poster or sign with a (QRC) QR image. (FIG. 2; 70). The QR code redirects a person using his smartphone camera to an online website portal. (FIG. 3; 11). The (user) may input their (user) information and medical history via their smartphone if needed (FIG. 2). The portal will also provide an option for which vision screening services the (user) would like to receive and pay. (FIG. 3).

The (user) may select from the possibilities of visual acuity screening, color vision screening, visual field screenings, or other types of screenings. One of the options is direct, on-demand telemedicine consultation. (FIG. 3). The telemedicine consultation may be with a telecommuting or remote health care provider in real-time or near real-time telemedicine screening or phone call. (FIG. 13; 205, 77) (FIG. 13A; 77, 76, 205, 42). All service payments may be via online portal e-commerce checkout, cryptocurrency wallet address, or any other types of payment systems. (FIG. 3).

The payment may be via a web portal or a mobile application on the patient's smartphone. After the (user) pays for the screening patient may enter the screening exam room. The patient may sit in the exam chair and put on the eye patch on the right eye (FIG. 4; 14, 15). The (assistant) virtual assistant, human assistant, or remote telemedicine health care provider will instruct the patient too look at a smaller vision acuity line of letters and speak the letters he or she can see (FIG. 7; 86, 24, 400). The exam room computers may have speech-to-text software that can be analyzed by artificial intelligence software. (FIG. 7; 400, 401, 44).

The AI computer will determine if the optotypes match with a specific visual acuity line. The AI software will recognize if the (user) has spoken the letters correctly (FIG. 7-400, 401, 14, 86, 24) (FIG. 6; 71, 24, 16, 86). In some cases, a virtual or non-virtual assistant or live eye doctor may also validate the accuracy of the visual acuity screening results. (FIG. 6; 80, 76). The option of having headphones is also possible for patient and remote health care practitioner. (FIG. 6; 76).

The patient will be instructed to start the color vision testing and after the visual field test. The artificial intelligence computers will record patient answer via speech to text (dictation software) and analyzed by artificial intelligent software to verify the patient accuracy. A visual acuity measurement will be provided using the percentage of accurate optotypes spoken. The calculation will determine a passing or non-passing vision screening result.

Furthermore, the (assistant) may validate if the patient answered correctly. Following this, the patient will cover the left eye and perform the visual acuity and color vision test again (FIG. 6; 71, 24). All information will be stored on patient vision screening, limited micro EMR, or EMR record, on the computers or the cloud (cloud computer) or annotated on a vision screening form by the (assistant) virtual or physical assistant (FIG. 6; 200). In the case of the (AIVA) providing the recording, it will be recorded via speech to text and analyzed with AI software for accuracy.

After the visual acuity and color vision screening is completed a visual field screening will be performed (FIG. 6; 72, 23, 16, 18). The patient will have his or her left eye covered with an eye patch testing the right eye vision field similar to a (confrontation visual field). The patient will press a clicker on the exam chair (FIG. 6; 23). The patient will be instructed by the computers to look at a fixation target; eye-tracking software will validate if he or she has moved from the eye fixation target. (FIG. 6; 24).

The patient will press the button every time the computers present an image on the large screen television (FIG. 6; 8, 23). After completing the visual field screening, the user will be instructed by (assistant) computers or virtual assistant or physician assistant to cover his or her right eye with the patch, and the visual screening will be performed the same way. (FIG. 6; 15). The visual field screening AI software will analyze and interpret the (user) visual field results to detect defects that can be caused by any neurological problems or ocular health problems (FIG. 8; 26, 27, 28, 14) (FIG. 9A; 1, 2, 3, 4, 29, 73). The (AIS) artificial intelligence software and machine learning software (ML) (AIML) will determine if the missing defect of visual field correlates with an existing database, sample dataset, blockchain dataset or online dataset of visual field defects. (FIG. 14; 505, 504, 507, 508)(FIG. 14B) (FIG. 9C; 73, 74, 75, 502, 501).

The machine learning—artificial intelligent software will also compare the visual field defects with a list of sample datasets based on diagnosis from past doctors and current health care practitioners' assessment diagnosis. List of systems that may be used: TensorFlow, Keras, Scikit-learn, Microsoft Cognitive Toolkit, Theano, Caffe, Torch, or any open-source, commercial, or non-commercial software. The artificial intelligence software and machine learning software can gather data and collect necessary variations of quadrant visual field defect and quantification of spots in the visual field defect to determine if the visual field defect can be caused by an eye health problem or brain health problem. A 3 point to 4 quadrant algorithm is used within the system to determine significant superficial changes. (FIG. 9C; 74) (FIG. 96; 500, 73, 1, 2, 3, 4).

The machine learning software may use (Bayes' theorem) equations to determine the percentage of probability of a brain or eye problem. (FIG. 14B). Bayesian learning treats model parameters as random variables in Bayesian learning; parameter estimation amounts to posterior computing distributions for these random variables based on the observed data. These same may be used to determine the probability of an eye-to-brain condition. This can be useful for diagnosing medical conditions such as concussion, accidents, contusions, pituitary adenoma, strokes, glaucoma, macular degeneration, and many more eye or brain conditions.

The database (sample dataset) of visual field defects may increase depending on the number of patient encounters in a plurality of locations worldwide. The database can be updated by connected health care practitioners with patient encounters of specific medical visual field defects and written diagnoses. After the conclusion of the vision screening, the computers will print out the results or email results with possible anomalies perceived. If a live assistant or if the health care provider is connected, they can write a vision screening report and go over the results. The system can also print out a visual screening report automatically if needed.

The information (screening report) may also be emailed to the user, DMV, primary health care provider, or eye doctor, or any other location if requested by the patient. The patient has the ability to request a live telemedicine consultation at the exam room if he or she needs a telemedicine consultation with any health care provider or (eye doctor) and more information on the vision screening results. (FIG. 13A; 205, 77, 42). In some cases, the patient may require a more extensive test with a virtual reality visual field analyzer (FIG. 11; 43) This more extensive test will be considered to have an extra cost to the patient in some cases.

The visual field virtual reality headset is needed to be position on the patient appropriately, and the virtual eye doctor or assistant may help with this (FIG. 10; 43, 200). A live health care provider (eye doctor) will appear in the exam room or on the patient personal smartphone or on the room displays, cockpit simulation frame computer, or tablet if needed and will be able to review all patient results and go over all needed information with the patient (FIG. 10-40, 42). AI and machine learning (AIML) software will be used to evaluate the results of the (VR) visual field screening. (FIG. 11; 509, 600, 601). The visual field software will be screen mirrored to a computer where the AI software can access the images and information for further analysis.

The telemedicine health care provider may also view the results via remote access software and control the system if needed. In some cases, a telemedicine health care provider can ask the patient if the user would like to perform a more thorough virtual reality visual field screening that may inquire more expenses. The payment can be made via a web link portal that can be scanned with a patient personal cellphone camera (QR code. (FIG. 10; 12, 40, 77, 80). This will be used for more in-depth vision screening analysis exams.

Any network health care practitioner (eye doctor) may control and screen view vision chart software, EMR, color vision software, pupillary distance software, confrontation field software, and virtual reality visual field equipment and software if needed. (FIG. 10; 40, 42, 600) (FIG. 13; 44, 401, 400, 77, 31, 24). Any network health care provider or (eye doctor) can diagnose the patient by viewing the visual field results and all the vision screening results. The virtual reality visual field tester (large screen simulation confrontational visual field and/or virtual reality visual field tester remotely are two tools that can be used for diagnosis and verification. (FIG. 10; 74, 40, 42). The network health care provider (HCP), (eye doctor) or (assistant) will be able to control the equipment remotely via his or her portable electronic device (PED) from anywhere around the world via a (Remote administration tool), (Remote Desktop tool) or (remote access software).

Remote administration tool (RAT) technology refers to any method of controlling a computer from a remote location. Remote desktop refers to a software or operating system feature that allows a personal computer's desktop environment to be run remotely on one system while being displayed on a separate client device. Remote access software remote control software, allowing view, control, maintenance of computers and other devices. The health care practitioner (eye doctor) can perform an automated static perimetry test, kinetic visual field test, frequency doubling perimetry, Amsler grid: A basic visual field test for central vision.

The eye doctor may select from any of the following 10-2, 24-2, or 30-2 visual field degrees temporal and nasal test. A 10-2: Measures 10 degrees temporal and nasal and tests 68 points. A 24-2: Measures 24 degrees temporal and 30 degrees nasal and tests 54 points. 30-2: measures 30 degrees temporal and nasal and tests 76 points. All are essential for a more detailed visual field screening analysis and may be controlled and viewed by the selected eye doctor anywhere in the world to help screen or diagnose a patient. The interconnection between the health care practitioner computer and all computers in the exam room will all interconnect via (Starlink internet), satellite internet, ethernet, 4G, 5G, 6G, or wifi. (FIG. 13A).

In cases of abnormal results, the eye doctor may remotely perform a realtime or near realtime tele-medicine remote visual test. The health care practitioner (eye doctor) may provide service with (RAT) remote administration tool technology to interconnect both systems. After the evaluation is complete, the health care provider or (eye doctor) may refer the patient to nearby emergency rooms (ER), medical doctors, or eye doctors by emailing a referral form to the patient with an included google map link or map link to the location. After the completion of the virtual screening and virtual reality visual field testing, the patient will receive results via email or printed format. The patient may also request the eye doctor or assistant to email send results to the patient-doctor of choice.

In some cases, renewal or prescription of medical devices such as eyeglasses can be done using the system. The future of this system will evolve with time. The (AI-VFTR) (Artificial intelligent visual field telemedicine room) system can add other medical equipment via upgrade adapters systems. The update system may have adapters on the simulation frame to provide adapting additional medical equipment.

The (AI-VFTR) may be upgraded to have a digital phoropter, automatic lens-meter, fully auto kerato-meter, fully automatic non-mydriatic retinal camera, fully automatic auto-refractor, fully automatic eye pressure tonometer. Adapters to the cockpit simulation frame can be position to secure all additional equipment. All equipment may be controlled by software and a health care provider controlling the software via remote administration (remote access, remote screen share, remote control) from anywhere in the world. All of this equipment will have software running on the computers in the system that can be controlled remotely via remote administration tool technology.

A second upgrade to the system is the addition of (medical images NFT). The computer systems may contain a second software that collects a sample dataset of vision screenings and visual field results. These sample datasets can process medical information and images as a non-fungible token (NFT) with the authorization of the (user) patient. The health care practitioner and (user) may receive royalties or commission for sharing their medical information, diagnosis and images. These NFT medical sharing sample datasets option may be driven by a blockchain smart contract or legal contract between the health care practitioner, the company providing the service, and the patient.

A non-fungible token (NFT) is a unit of data stored on a digital ledger, called a blockchain, that certifies a digital asset to be unique and therefore not interchangeable. NFTs can represent items such as photos, videos, audio, and other types of digital files. NFTs are tracked on blockchains to provide the owner with proof of ownership.

### BRIEF DESCRIPTIONS OF THE DRAWINGS

FIG. 1 Illustrates a work flow diagram of vision screening and visual field analyzing screening exam system in a room inside, a mall, shopping plaza, medical facility, building, or outdoor or anywhere. (Kiosk, booth or room).

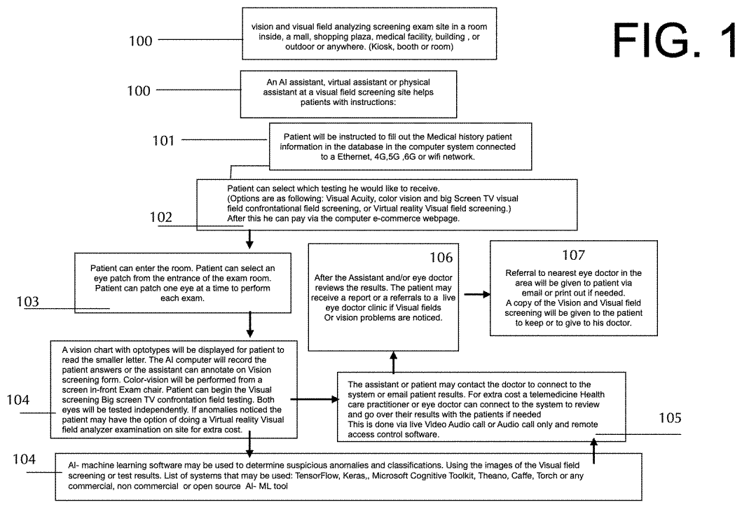

FIG. 2 illustrates a pictorial diagram of the (Kiosk, booth or room) in a a mall or shipping mall with sign up portable computers (laptop), an entrance sign with a door with smart locks, QR code, computer camera, QR code scan sign, eye patch box, a screen with a virtual or AI assistant, an human assistant, a user with an eye patch on one of his eyes, a rollin chair and a sign on the top of the (Kiosk, booth or room).

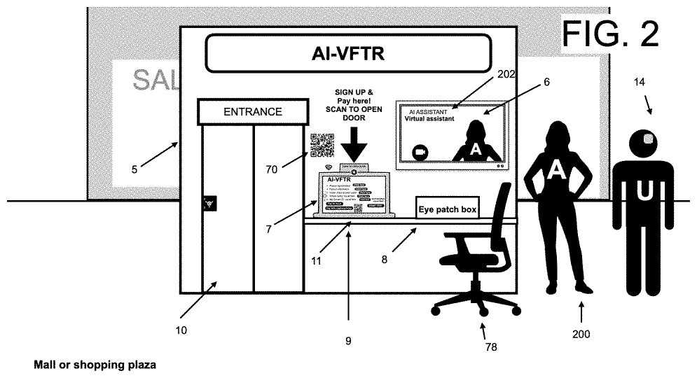

FIG. 3 Illustrates a pictorial diagram of the sign in computer (Which can be laptop, tablet or user personal smartphone). The sign in webpage is running on a browser or kiosk software, the sign in website will have a section for person appointment information, personal information (user medical history form), Vision check, color vision screening, virtual reality visual field, large screen monitor visual field testing, payment link that includes credit card or cryptocurrency or any other payment system. A link button to a start test indicates that the session may start.

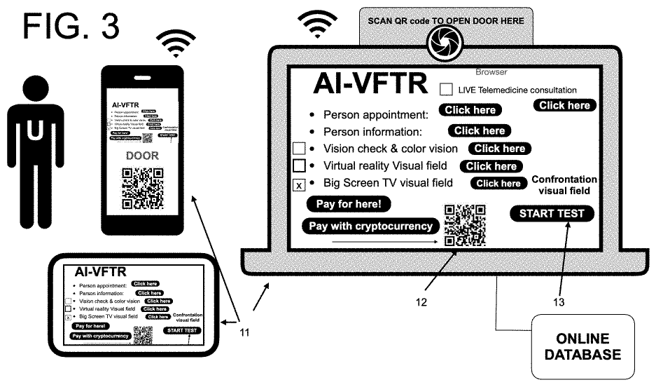

FIG. 4 Illustrates pictorial diagram embodiment external part of an exam room inside a building. The door, a display monitor with a virtual assistant speaking instructions, a user putting on an eye patch, a user withe his or her smartphone with a Qr code to scan onto the computer camera to open the smart lock doors to access the in side of exam room. All computers and displays interconnected with hotspots, internet ethernet, wifi, 4G, 5G, 6G or any other wireless networks.

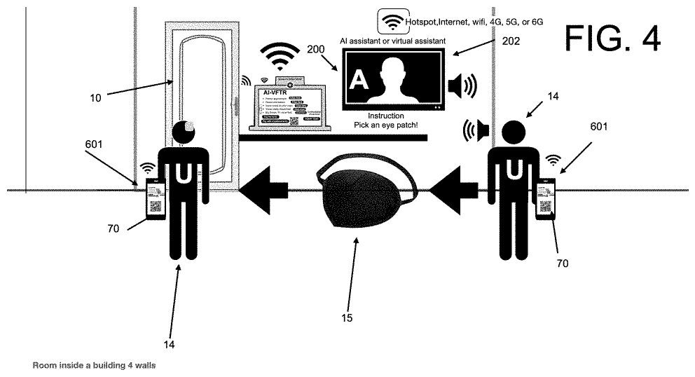

FIG. 5 Illustrates pictorial diagram embodiment an human physical assistant outside of an exam room. The design a user sitting in an exam chair with a large screen monitor or smart television in-front of the user. The user is speaking and the tablet computer running multiple software is connected to a direct call to a (assistant or health care provider).

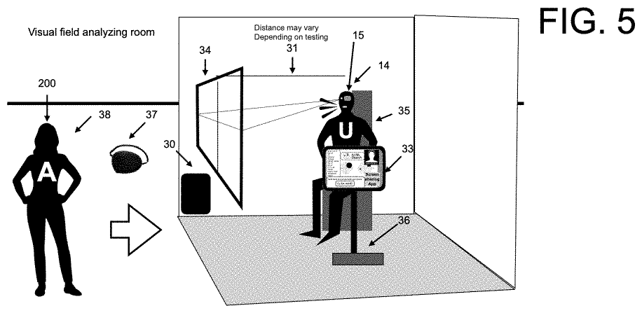

FIG. 6—Illustrate a pictorial diagram embodiment of different scenarios inside of the exam room with the displays in-front of the user where the user has their eye patched and is connected via teleconference or audio call to an (assistant) or (health care provider). Where the Assistant or health care provider is viewing via screen share technology the remote (AI-VFTR) computer tablet testing and results of the visual field and color vision screenings using wifi, 4G, 5G, 6G to interconnect both systems. Where the health care practitioner is on a video call also to achieve more clear audio connections. Where a Qr code is displayed on the tablet computer next to the user where the user can scan to achieve a direct connection via a direct call to the health care provider.

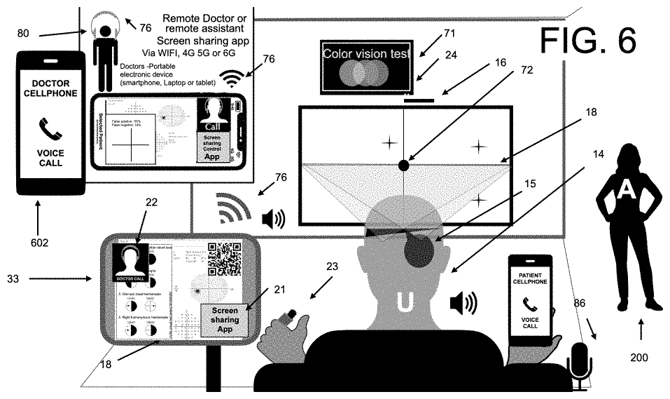

FIG. 7—Illustrate a pictorial diagram embodiment of different scenarios inside of the exam room with a displays in-front of the user. Where the displays top is a vision chart with multiple letters and the bottom display is a simulation confrontational visual field test. Where the user will state the smallest letters they can see and the computer running an AI computer and speech to text software will calculate the (users) visual acuity machine the smaller line spoken to the Visual acuity stated. Where the user visual field results are displayed on to the computer next to the user and the remote health care practitioner can view via screen share the results and go over result via a voice call between the health care practitioner and the (users) cellphone.

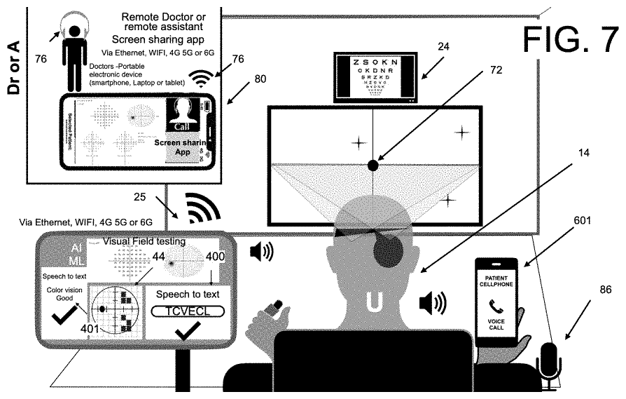

FIG. 8—Illustrate pictorial diagram embodiment of a group of sample data sets of visual field defect and diagnosis that are used for the artificial intelligence and machine learning software inside the computers to determine the type of visual field defect and if its eye or brain related.

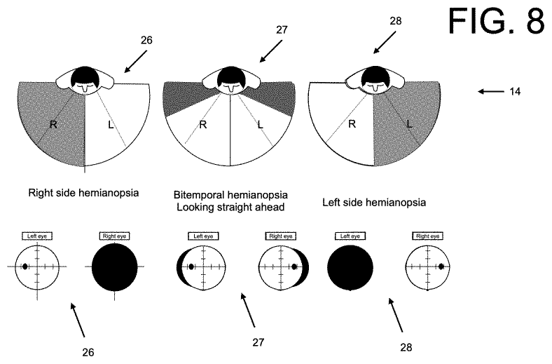

FIG. 9—Illustrate pictorial diagram embodiment of a group of sample data sets of visual field defect and diagnosis that are used for the artificial intelligence and machine learning software inside the computers to determine the type of visual field defect and if its eye or brain related.

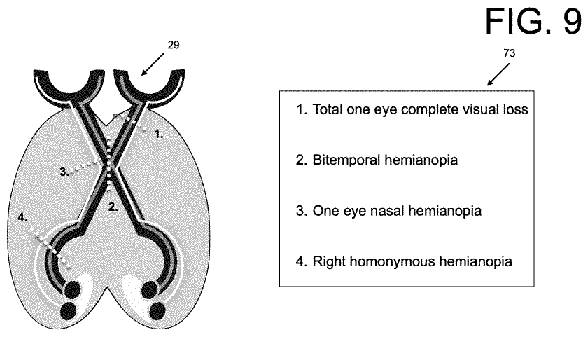

FIG. 9A Illustrate pictorial diagram embodiment of a group of sample data sets of visual field defect and diagnosis that are used for the Artificial intelligence and machine learning software inside the computers to determine the type of visual field defect and if its eye or brain related.

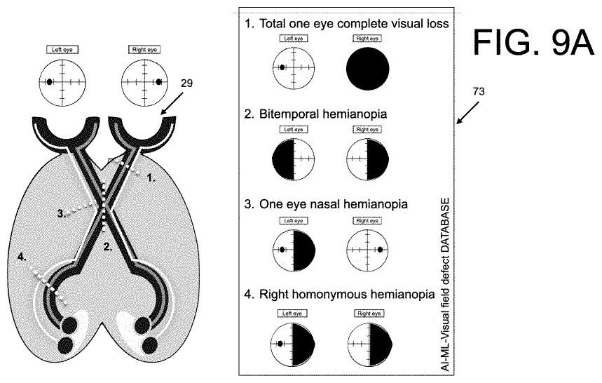

FIG. 9B Illustrate pictorial diagram embodiment of a group of sample data sets of visual field defect and diagnosis that are used for the Artificial intelligence and machine learning software inside the computers to determine the type of visual field defect and if its eye or brain related. Where the artificial intelligent computer can use the data set via a 4 quadrant A, B, C, D algorithm to provide probable diagnosis using machine learning.

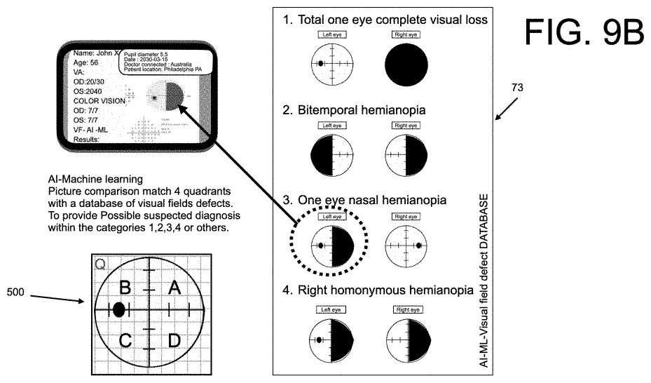

FIG. 9C—Illustrate pictorial diagram embodiment of a group of sample data sets of visual field defect in the artificial intelligent software and its algorithm to determine possible diagnosis via a 3 point 4 quadrant algorithm in each eye and compare to sample data set.

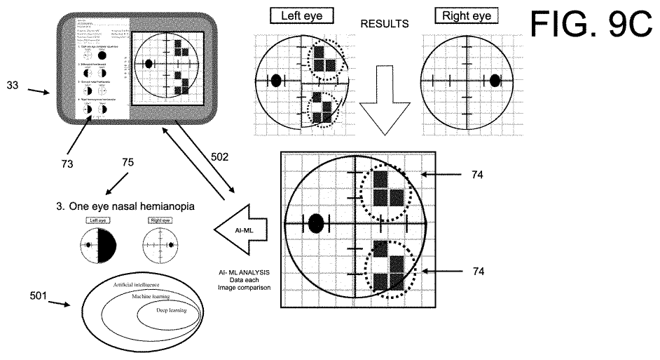

FIG. 10—Illustrate a pictorial diagram embodiment of different scenarios inside of the exam room with a displays in-front of the user. Where the displays top is a simulation confrontational visual field test results with superior temporal defects. Where the health car practitioner is connected with the user via cellphone call and via screen sharing and control to the tablet inside the exam room. Where the health care provider is requiring a more precise virtual reality visual field test to determine if the visual field defect is correct and needs a referral or further evaluation. Where the user via cellphone will provide the health care provider a code to access the computers systems in the (AI-VFTR) and a payment link is seen on the computer next to the user to submit payment or insurance information. Where the results are screen mirrored to the screen for explanation, and counseling. Where all the connection in the system and the health acer provider are done via internet via ethernet, wifi, 4G, 5G or 6 G cellphone wireless networks.

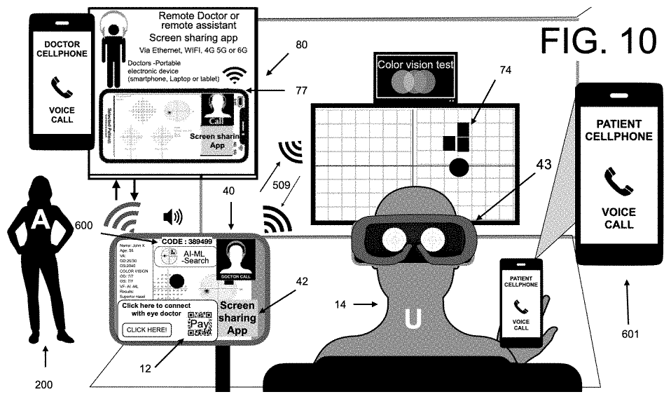

FIG. 11—Illustrate pictorial diagram embodiment of a virtual reality head set running a software to provide visual field analysis to a user. Where the virtual reality headset has the capabilities to screen mirror the information and test to a computer and can be viewed by a remote health care provider via (remote administration tool technology) or remote screen share and control technology. Where results can be printed inside the room.

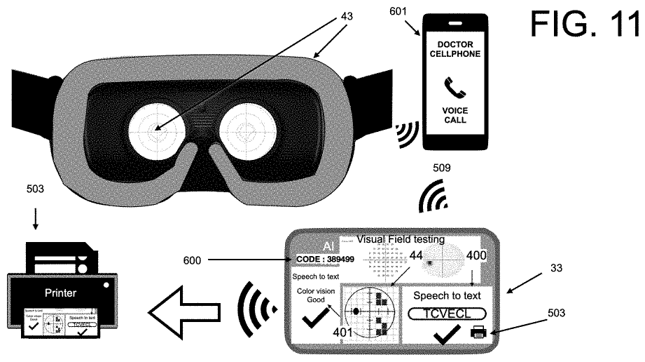

FIG. 12—Illustrate pictorial diagram embodiment of a types of designs proposed where the front view has a door entrance of the (kiosk, booth, or exam room). Where the side view may or may not have a window with smart glass, Where the back view are made of multiple glass doors that have smart glass and/or curtains, where the top view may or may not have a roof. Where the roof design has two vents to bring fresh air inside the room.

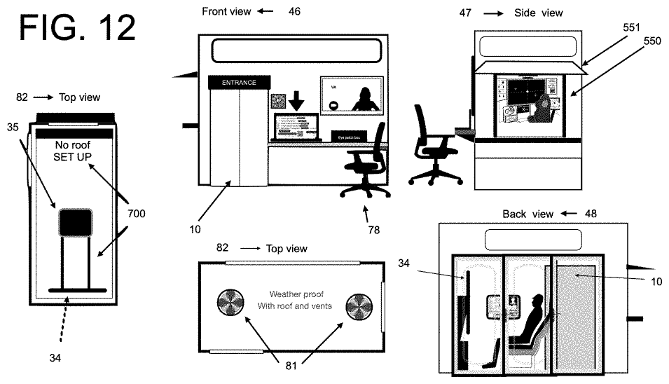

FIG. 13—Illustrate pictorial diagram embodiment Illustrates the back view of the (kiosk, booth or exam room) where the you can see a user if the curtains or smart glass are not activated. Where the person is sitting in an exam chair with a cockpit simulation setup system in-front of the user. Where the human assistant can fit behind the user. Where the room may have extra displays where the doctors image can be screen mirrored. Where the user is talking to the health care provider. Where the user is inputting his or her information inside the computers system providing a code to screen share their information with the health care provider. Where the health care provider laptop is viewing the an emulated screen of the computer inside the (AI-VFTR). Where the health care provide can view and control the (AI-VFTR) computers using ethernet wifi, cellular wireless connection 4G, 5G, 6G to interconnect both systems.

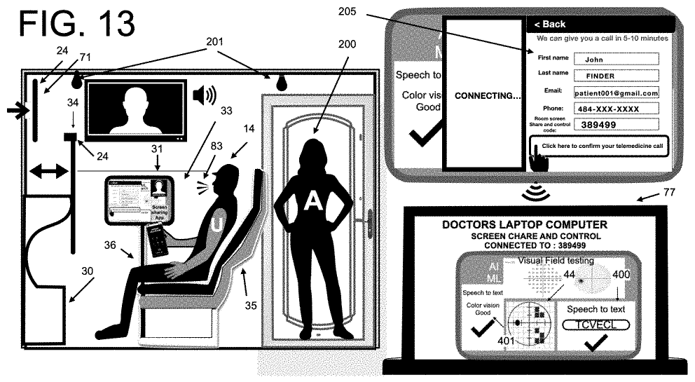

FIG. 13A—Illustrate pictorial diagram embodiment of a scenario where the user inputs his or her information on the (AI-VFTR) computers. This sends a message to an health care provider within a network with the information to call this phone number to provide on demand telemedicine service. After the health care provider call is established the user will provide the code to connect to the (AI-VFTR) computers systems via screen share and control) via the health care providers personal electronic device (PC, smartphone, laptop or tablet).

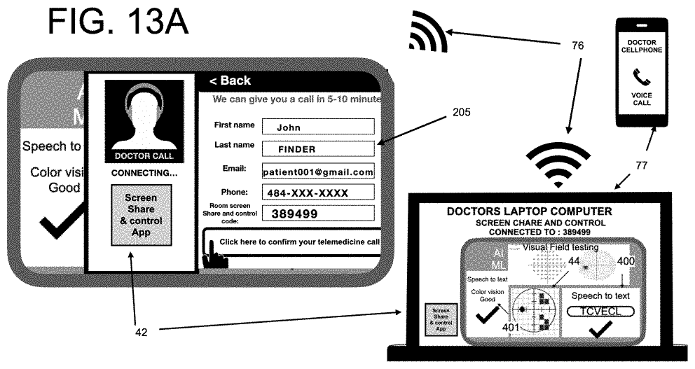

FIG. 14—Illustrate pictorial diagram embodiment of a (AI-VFTR) simulator cockpit display system assembled. The simulator cockpit frame holds all displays, large screen display, small screen display, eye tracking hardware, a large tablet computer with its display, a virtual reality charging and storing section, a printer, signs on the side of the large display, and the adjustable exam chair.

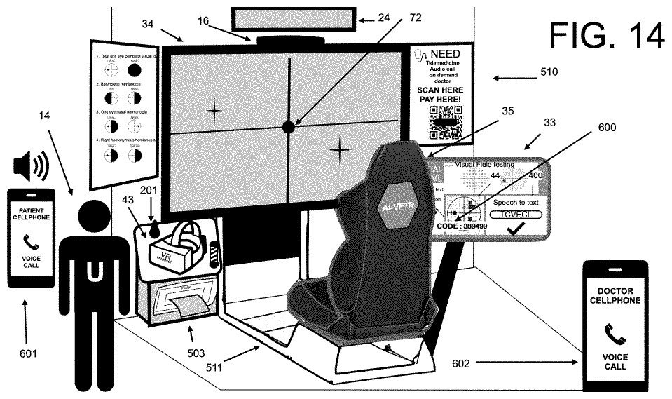

FIG. 14A—Illustrate pictorial diagram embodiment Illustrates a group of sample datasets diagnosis sets of visual field defect in the artificial intelligent software and its algorithm to determine possible diagnosis via a 3 point 4 quadrant algorithm in each eye and compare to sample data set for each eye.

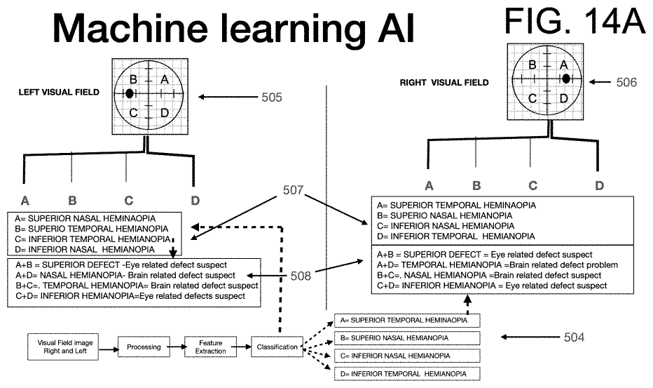

FIG. 14B—pictorial diagram embodiment Illustrates scenarios using the algorithm and AI machine learning technology where the system can determine if the visual field defect is brain related or eye related via quadrant pattern recognition, analysis, statistics, datasets and probability.

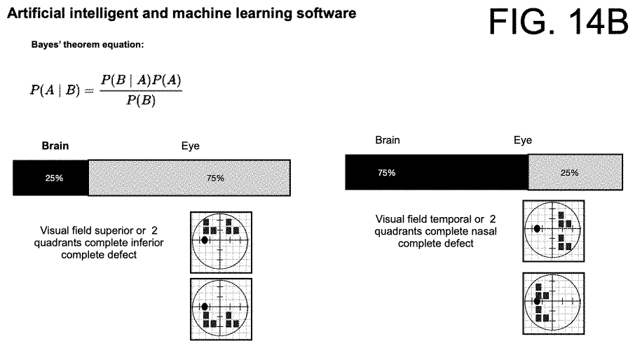

What is claimed is:

1. A method, system, apparatus and workflow of one or a plurality of (AI-VFTR) (Artificial intelligent visual field telemedicine rooms) systems that can be set all over the world and anywhere in the world. The (AI-VFTR) system providing an on demand neurological and visual health screening and vision screening system to any user including a booth, kiosk, and/or exam room with a display screen simulator cockpit set-up, automated large screen visual fields setup, virtual reality visual field testing, visual acuity testing, color vision testing, pupillary distance and medical data analysis and diagnosis using artificial intelligent-machine learning software and with the capabilities for providing an on-demand telemedicine consultation by a health care provider who can connect to the system via video audio, audio, audio phone call, remote administration tool technology, remote screen share and control access tool technology to computers systems inside the (AI-VFTR) to provide vision health service to patients (user) comprising:
a room structure design that can be a booth, kiosk and/or exam room.
a assistant that may be a physical person, virtual medical technician, physical technician, telepresence technician or may also be an artificial intelligent virtual assistant.
a simulator cockpit or cockpit simulator frame has monitors or screens attached with an adjustable seat.
a sign-in webpage on a browser on a laptop computer outside of the room.
a remote connection to an on-demand health care provider that may provide a connection from a one or more network of health care providers to the patient in the room equipment for counseling, diagnosis, review of testing, screening results, medical device prescription and referrals.
a computer system that will run visual field software, vision acuity software, color vision screening software, eye-tracking software, pupillary distance software, remote administration tool software, and artificial intelligence machine learning software.
a speech to text software to a data collecting system where the patient can speak the letters, numbers or symbols, (optotypes) from the vision acuity and color vision screening chart, and the information via speech to text can be transferred to the database for machine learning software to compare to results.
a visual field software and clicker to provide visual field testing via simulated confrontational field using a cockpit simulator frame with monitor screens and second via virtual reality headset both with a wireless or non-wireless clicker.
a eye tracking device and software working with the visual field software and vision screening software to determine eye fixation, pupillary distance and eye movements, and to maintain desired fixation point.
a vision chart and color vision software designated for analyzing person, patient, or customers vision acuity and color vision deficiencies
a machine learning artificial intelligent software gathering data software and analyze data from the results of speech to text color vision, visual acuity, and visual field testing images results.
a large and small screen monitor or television mounted on a (cockpit simulator frame) to perform vision screening and testing using a simulator cockpit frame.
a printers used to print out screening results, referrals forms to a health care provider, medication prescriptions, medical device prescriptions, receipts for service, or any other medical intended paperwork needed.
a internet connection used to connect all of the computer systems inside and outside the (AI-VFTR).
a virtual reality headset to run a software to deliver visual field testing to both eyes independently.
a remote administration tool used to secure remote control, remote access, remote screen share from a health care practitioner and any computer in the (AI-VFTR)
a doors be made with or without (smart glass) using a (smart film) on the glass doors for privacy and reducing lighting in the room.
a uv disinfecting system or disinfecting procedure to disinfect equipment after every session.
a sample medical image dataset

2. The room of claim 1, wherein the room structure design including four walls, floor, vents, roof with one or multiple doors, curtains or smart glass technology, and/or window openings depending on design. The doors can open and close remotely or physically. Where the doors and windows may be locked remotely or physically. The room structure can have a roof with vent or not have a roof depending on design of the room and maybe powered by external power source with electric sockets and plugs. Where the room may be built to be a permanent or temporary booth, kiosk and/or exam room in a larger room or leasing space in a building. Where the room can be a room inside any building or structure. The (kiosk, booth, or exam rooms) can be installed in a plurality of location anywhere in the world. Where in the booth, kiosk or room may be inside or outside of any building, hospital, department of motor vehicles building, inside military bases, overseas military bases, government facilities, malls, shopping plazas, watercraft ships, trailer, kiosk, booth, or any location that include medical equipment of any specialty anywhere in the world. The (kiosk, booths or exam rooms) are similar but larger than photo-booths and can hold one to three people inside but can vary depending on design location. all equipment may or not be built into the room but built on a cockpit simulation frames, stands, and furniture inside the (kiosk booth or exam rooms). The designs of the rooms, booth and kiosk may vary depending on setting and models. Some kiosk or booth designs may fit almost side by side (modularly) depending on design.

3. The assistant of claim 1, wherein the assistant may be a physical person, virtual medical technician, physical technician, telepresence technician, or may also be an (artificial intelligent) virtual assistant. In some cases a (health care provider) (eye doctor) may be an assistant. Any of all the assistant types may speak, provides instruction, collects data results, sends and receives data and/or responds to questions.

4. The simulator cockpit or cockpit simulator frame of claim 1, wherein the cockpit simulator frame has monitors or screens attached with an adjustable seat. The cockpit simulator frame provides a proper working distance to evaluate all vision examinations. The televisions or monitors used to (screen share) the data to one or more monitors, screen, computer screen, computer tablet screen, or virtual reality headset. The cockpit simulation frame may have additional adapting frames or furniture to upgrade the with other ophthalmic or medical equipment in the future.

5. The sign-in website of claim 1, wherein the sign-in website runs on a browser on a laptops computers, tablet computer, webpage, or mobile application. The computers may be inside or outside of the room. The sign in website can be access via QR code to do it via patient or customers own cellphone via web portal or mobile application. The computers may also run kiosk software or a webpage with e-commerce payments. The sign in portal may be used to gather patient information (medical history information), chief complaint and payment information. This information is inputted by patient via the sign in computers or customers, any person, (patient own personal electronic device via webpage or mobile application). Payments for medical service and screenings can be made via sign in laptops, tablets or mobile application or weblink with credit card shopping cart, self-checkout, central bank digital currencies, or cryptocurrency address. The system can also gather data for health insurance information and bill patient if needed. The medical screening service to be rendered prices will be displayed on the website, mobile application and or kiosk software. In some cases the payment can be waived for no cost service in humanitarian missions or free vision or medical screenings.

6. The remote connection to an on-demand health care provider of claim 1, wherein the remote on-demand health care providers may provide counseling, diagnosis, medical device prescriptions, review of testing results, and referral's to patient via plurality of (AI-VFTR) systems around the world. That the health care provider can be anywhere in the world and can provide service from anywhere in the world to any (AI-VFTR) (Artificial intelligent visual field telemedicine room) (kiosk, booth or exam room) anywhere in the world. That the health care provider can connect to the system and provide telemedicine services to a person via the cockpit simulation frame system computer system, display or (patient personal portable electronic device). Where the patient can screen mirror his cellphone video and audio to any displays in the room or cockpit simulation display. The telemedicine connection can be via tele conference video audio, audio only, text messaging, email, personal cellphone voice call, cellphone application for telemedicine service and/or remote screen share and control, remote administration tool technology or combination of any of the following. That the remote control to the medical software can be controlled using any communications technology service provided by any companies or software that may provide video-telephony, online chat services, teleconferencing, telecommuting, and remote control screen distance control to all medical software that controls the medical equipment in the room. That all telemedicine service interconnections, remote control, and remote screen share connections are done with internet connections such as ethernet, wifi, 4G, 5G, 6G or better cellular wireless networks. All interconnection will be done using the doctors, (AI-VFTR) computers and patients portable electronic device. The remote control and remote screen share will are done with (remote administration tool technology, remote access and control and cloud computer technology). All patient information can be access and updated remotely. Where the connection from the health care provider and the (AI-VFTR) system can be achieved via a (remote administration tool technology), remote access software, remote control and screen access on the (AI-VFTR) computer systems to screen share to view patient data and results. Where in the health care provider can connect to the system via remote administration tool technology, remote access control screen share technology, telemedicine video audio technology, chat and audio phone call. Where the health care provider may achieve connections with the patient via the patient personal cellphone via a direct link QR code and where the health care provider may be any type of health care provider including medical doctor, optometrist, ophthalmologist, neurologist, nurse, physician assistant or any other types of health care providers with a valid license to practice as a health care provider. Wherein the health care provider may forward information, referral forms, prescriptions or visual screening results to any health care provider, medical device provider, optical stores provider, ophthalmic medical device provider, Department of motor vehicles office, medical specialist or patients (customers). Where the health care provider may be part of a network of health care providers that are assigned to provide on demand medical service.

7. The computer systems in claim 1, wherein said plurality of computers in the (AI-VFTR) may be running on any operating system linux, IOS, windows, chrome os, raspberry pi, android and/or Mac. Where in the some or all the computer may run multiple softwares, virtual boxes and/or emulators at a time. Where the computer systems be used with screen mirror software via wired HDMI, bluetooth or wifi. The screen mirror and control may be done via a screen mirroring software that allows control and view of all the computers software to be viewed on a secondary computer via that can be controlled from the background. The computers may run virtual boxes with different operating systems (OS) in some situation to run intended software needed to perform the examination. The computer systems may run visual field software, kiosk software, vision acuity software, color vision screening software and artificial intelligence machine learning software to interpret results and gather information for probable suspected neurological or ophthalmologic vision psychophysical diagnosis including contrast sensitivity testing. Where in all the computer systems in the (AI-VFTR) may be controlled remotely via a (RAT) remote administration tool technology and/or any remote access control tool technology from any open source software or company providing (RAT) technology service software. The computer remote control and visualization and connectivity may be achieved by any designated health care practitioner or technician (Assistant) in any remote or non remote location. The artificial intelligence and machine learning software may use (Bayes' theorem) to determine the percentage of probability of a brain and eye problem. Where sample dataset of information and patient diagnosis may or mayn't be gathered by machine learning software to learn for future deep learning and a future more precise diagnostic tool.

8. The speech to text software in claim 1, wherein the speech to text software can run on the computers systems on the (AI-VFTR) and are used to as data collecting system where the patient can speak the letters, numbers or symbols, (optotypes) from the vision acuity and color vision screening chart and the information via speech to text result data can be transferred to the artificial intelligence machine learning software to compare to results. If the patient passes all of the results, the machine learning (AI) software, health care practitioner, physical or remote assistant may designate a passing rate depending on results (percentage correct). Where the speech to text software may be of open source, commercial or non-commercial source and the machine learning (AI) software may be of open source, commercial or non commercial source. All data and information data may be stored on the computers, servers, and/or cloud computer technology.

9. The visual field software and clicker in claim 1, wherein said plurality of visual field softwares may be displayed via screen share to the cockpit simulator frame display screen from a computer. Where the visual field softwares can be an application from a tablet in some cases running a kiosk software. The clicker can be bluetooth, wifi, or wired connection to the (computer running the visual field software). The visual field software consists of two different or same software running on two different devices a tablet or computer screen mirroring to a large display screen (television or monitor) with high resolution and/or a virtual reality headset with two oculus. The virtual reality headset and its software can be of open source, commercial or non-commercial means. Where in the visual field software projected on the display screen can be of open source, commercial or non-commercial means. The visual field software may be two independent software running on two different devices and/or same software running on both devices. The visual field software may be a mobile application screen mirrored to the virtual reality head set or a visual field mobile application screen mirrored to the large display in-front of the person receiving the service. Where the visual field software is calibrated with the large screen display to provide accurate examination and distance for all degree of visual fields.

10. The eye tracking device and software systems in claim 1, wherein the eye tracking device and softwares work with the visual field software, color vision software and vision acuity software to determine eye fixation and eye movements away from the central fixation point. The eye tracking hardware and software may use infrared technology, cameras depending on the eye tracking software and hardware used. The eye-tracking devices and/or softwares may run inside the computer system. The eye-tracking device and software may be from an open source, commercial or non-commercial means. The eye-tracking software may also provide eye movement measurements (extra ocular muscle testing) and can also be configured to provide approximate or exact pupillary distance in some cases by using specific software, mobile applications, artificial intelligence and/or machine learning software. In the case of pupillary distance measurement the system may require an extra payment for the printout, email or text message for customers pupillary distance measurements.

11. The vision chart and color vision software in claim 1, wherein the vision chart software and color vision software designated for analyzing patient or customers vision acuity and color vision deficiencies. The vision chart and color vision software work with a speech-to-text (dictation) software, artificial intelligent and machine learning software to determine if the patient speaks the optotypes (letters, numbers, or symbols) correctly to gather optimal visual acuity screenings and color vision screenings. The system may be configured with a translator for multi language settings or recognition. Where the patient can read the lowest selected (optotypes) on the screen and the artificial intelligence computer can categorize the letter to determine the best corrected visual acuity measurements for both right and left eye. Where the patient can read the numbers or figures on the color vision chart and the artificial intelligence software inside the computer can determine visual screening results.

12. The machine learning-artificial intelligent softwares in claim 1, wherein the machine learning-artificial intelligent softwares may run on all computers (laptop, tablet, virtual reality headset) and to gather data from the results of speech to text software, color vision software, visual acuity software, pupillary distance software and visual field software testing images. There could be multiple AI machine learning software running inside the computers and used to gather data and sample dataset in different forms for future deep learning studies. The machine-learning artificial intelligence software will use the images of the visual field screening or vision screen test performed to determine if they are brain or eye-related via a black on white, 3 or more point to 4 quadrant system algorithm. The machine learning-artificial intelligent software will also compare the visual field defects with list of samples datasets based on diagnosis from past doctors and current health care practitioners assessment diagnosis. List of systems that may be used: TensorFlow, Keras, Scikit-learn, Microsoft Cognitive Toolkit, Theano, Caffe, Torch or any open-source, commercial, or non-commercial software.

13. The large and small screen monitors or television in claim 1, wherein the big screen monitors or television may be mounted on a (cockpit simulator frame) or wall. Where the screen can be high resolution (4K) or better flat screen and can be of Smart TV, 3D, QLED, HD, UHD, OLED, LED, LCD or any type of display device and technology. Where the screen monitor or television will be connected to all vision testing software via HDMI wire, bluetooth or wifi to screen mirror. Where the distance is calibrated to determine visual acuity measurement for optotypes to gather visual acuity, color vision and visual field testing. The screen may have adapters that can move the screen to desired position for calibration.

14. The printer of claim 1, wherein the printers may be of any of the different types of printers include inkjet printer, laser printer, thermal printer. The printer can be color black, solid ink, thermal printer, and white printer. The printers can used to print out visual screening results, pupillary distance measurement, referrals forms to health care providers, medication prescriptions, receipts for service or any other medical intended paperwork needed. The printers can be wireless or non-wireless (wired) to connect with the computers. The printers can be used physically or remotely by an assistant or by a health care practitioner to print out any document remotely by controlling the software via (RAT) remote administration tool technology and/or (RAS) remote access and control software.

15. The internet connection of claim 1, wherein internet connection is used to connect all computer systems inside the (AI-VFTR) to the remote health care practitioner via ethernet, hotspots, wifi, 4G, 5G 6G cellular networks, satellite internet, starlink satellite internet connection or more advanced cellular networks. All internet connection can be used to provide realtime or near realtime connection with the health care practitioner to the exam room computers. The tele medicine interconnection between the health care practitioner to the (kiosk, booth or exam room) or patient phone may be via phone audio call, Audio video call, Audio video application software, remote administration tool technology, remote desktop control technology to achieve by a remotely connected health care provider.

16. The virtual reality headset of claim 1, wherein the virtual reality headset runs software to deliver visual field testing 24-2, 10-2, 30-2, and/or central visual field testing. The virtual reality headset can be connected to a computer with can screen mirror the information and result on to it. The information can now be view from the remote health care practitioner via remote access or screen sharing tool if needed during or after the test is done. The virtual reality headset visual field software may be of open source, commercial or non-commercial source. The images gathered from the virtual reality headset visual field testing may be analyzed by a remotely or non remotely connected health care provider. The images gathered from can also be analyzed by the AI machine learning software running on the computers. The virtual reality headset may be commercial or non-commercial hardware. The virtual reality head set may also be a smartphone running software for visual fields testing attached to a virtual reality headset smartphone adapter. Some VR headsets also have eye-tracking sensors and gaming controllers. The current headsets may or may not also have eye-tracking sensors. The VR headset can also have a wired or bluetooth connected clicker or gaming controller for the person to click during the examination or screening.

17. The computer systems in the (AI-VFTR) of claim 7, Wherein all the computer systems in the (AI-VFTR) may be controlled remotely via a (remote administration tool) remote desktop control access tool by any designated health care practitioner or technician (Assistant). Where a database of information and patient diagnosis may or mayn't be gathered by machine learning software to learn for deep learning and a future more precise diagnostic tool. That video voice or voice calls can be done via the computers on the systems if needed. That video or voice calls can also be done via the health care practitioner and the customer (patients) personal electronic device (laptop, cellphone, smartphone, or tablet).

18. The remote administration tool of claim 1, wherein the remote administration tool (RAT), (remote desktop software) or (remote access and control software) application can be of open source, commercial or non-commercial source. The remote administration tool (RAT) can be used to secure a remote control, remote access, remote screen share to any computer in the (AI-VFTR). Remote administration refers to any method of controlling a computer from a remote location. The interconnection of both systems can be done from any assigned health care practitioner with a remote administration support tool software from anywhere in the world to the (AI-VFTR) computer system via a secure encrypted connection via ethernet, wifi, satellite internet, starlink satellite internet connection, 4G, 5G, 6G or more advance wireless or cellphone wireless networks. All computer with softwares, vision screening software and hardware may be control via remote administration tool (RAT), (remote desktop software) or (remote access and control software) technology by an (assistant) or (health care provider).

19. The visual field screenings of claim 9, clients requesting visual field screenings may be present in any building, hospital, Department of Motor Vehicles building, inside military bases, overseas military bases, government facilities, malls, optical shops, pharmacies, optometric offices, ophthalmology offices, watercraft ships, trailer, kiosk, booth or any location that include medical equipment of any specialty anywhere in the world. The visual field screenings system may relate to preventive medicine, neurological examination or ocular health examination, or any other medical field test. In certain embodiments, the vision field screenings system may also facilitate the treatment or diagnosis of medical condition by a remotely connected health care provider connected via a remote access control and sharing software to the system or receiving the results via email, paper, or secure file transfer.

20. The doors of claim 1, wherein the doors may be made of (smart glass) using a (smart film) on the glass doors to adjusting light transmission between transparent, opaque, or photochromic using AC power electrochromic. The smart glass can be activated automatically remotely to prevent glare during the screenings. Some room designs may or may not have smart glass door and smart windows depending on the design used for the type of booth, kiosk or exam room. The smart glass may be used UV protection.

21. The room of claim 1, wherein the room structure including a eye patch box or vending machine inside or outside of the room. The room has internal medical equipment such as multiple computers, vision screening chart software, color vision chart software, exam chair, chairs, eye tracking hardware, eye tracking hardware software, security cameras, infrared cameras, computer cameras, microphones, cockpit simulation frame, screen display monitors or televisions, eye examination equipment, artificial intelligence software. Where the rooms can be equipped or upgraded to have any medical equipment such as automatic digital phoropter, fully automatic lensmeter, fully auto keratometer, fully automatic non mydriatic retinal camera, fully automatic auto refractor, fully automatic eye pressure tonometer and/or any ophthalmologic, optometric and neurological examination equipment with its control software running on the computers systems that can be controlled remotely.

22. The sample medical image dataset softwares in claim 1, wherein the said plurality of softwares collect sample dataset of visual screening and visual field results that can be processed as a medical non-fungible token (NFT) where health care practitioner and patient may receive royalties or commission for sharing their medical information, diagnosis and images. These NFT medical sharing sample datasets option may be driven by a blockchain smart contract or contract between the health care practitioner, the company providing the service and the patient. A non-fungible token (NFT) is a unit of data stored on a digital ledger, called a blockchain, that certifies a digital asset to be unique and therefore not interchangeable. NFTs can be used to represent items such as photos, videos, audio, and other types of digital files. Access to any copy of the original file, however, is not restricted to the buyer of the NFT. While copies of these digital items are available for anyone to obtain, NFTs are tracked on blockchains to provide the owner with a proof of ownership that is separate from copyright.
